(19) United States
(12) Patent Application Publication
(10) Pub. No.: US 2012/0258162 A1
(43) Pub. Date: Oct. 11, 2012

(54) CONTROLLED DELIVERY OF MOLECULES FROM A BIOINTERFACE

(75) Inventor: Mark A. Tapsak, Orangeville, PA (US)

(73) Assignee: Empire Technology Development LLC

(21) Appl. No.: 13/202,682

(22) PCT Filed: Apr. 7, 2011
(86) PCT No.: PCT/US2011/031604

§ 371 Date: Aug. 22, 2011

(51) Int. Cl. A61K 9/00; A61P 31/00; A61P 23/00; A61P 7/02; A61K 31/80; A61P 9/00; A61P 7/00; A61P 29/00; A61P 37/06

(52) U.S. Cl. 424/424; 424/78.17

(57) ABSTRACT

Disclosed are methods and apparatuses for delivery of bioactive molecules. The drug delivery systems include an implantable medical device which significantly reduces or suppresses adverse biological responses associated with implantable devices and also promotes vascularization in tissues surrounding the implanted device. The disclosure also relates to drug delivery systems designed to vary the rate of delivery of bioactive molecules with a change in the physiological environment.

### BACKGROUND

The following description is provided to assist the understanding of the reader. None of the information provided or references cited is admitted to be prior art to the present invention.

Implantable medical devices are often used for delivery of an agent, such as a drug, to an organ or tissue in the body at a controlled delivery rate over an extended period of time. Implanted devices are often associated with undesired biological responses such as inflammatory responses, foreign body reaction (FBR), fibrosis, cell adhesion, restenosis, calcification etc. Inflammatory response is caused by the tissue injury that results from implantation of the device as well as the continual presence of the device in the body. When a tissue is injured by device implantation, a wound healing response is initiated through a series of complex events.

Angiogenesis, the formation of new blood vessels from existing ones, is an important event in several biological processes, including wound healing. It plays a key role in determining the final functionality and integration of any implanted medical device. The controlled growth of vascular networks requires the timed release of multiple growth factors or angiogenic molecules. Some medical devices require close vascularization and transport of solutes across the device-tissue interface to ensure proper functioning of the device. However, because of biological responses such as inflammation and FBR, implanted devices tend to lose their function within the first few days to weeks following implantation. Some efforts, aimed at increasing local vascularization at the device-tissue interface, focus on the release of growth factors at the device implantation site for the purpose of inducing blood vessel growth. Few of the known techniques involves use of a biocompatible coating material which provides a slow release of angiogenic molecules, anti-inflammatory drugs and other agents through the coating on the implant. Indeed, a selective and sustained diffusion of various agents through a single coating material or membrane is often difficult owing to factors such as varying pH of the physiological environment in which the device is placed

Further, it has long been recognized that the materials commonly used to construct implantable medical devices stimulate an inflammatory response. Upon implantation, all foreign material is detected as such and a set of responses is triggered in reaction to the wound and chronic presence of the implanted material. The initial response is termed the acute inflammatory phase. Interestingly, it has been reported that during this period, the localized pH can reach levels as low as six. Over the course of days to weeks, the second phase of inflammation begins. Macrophage and lymphocyte cells predominate during this period. Ultimately, permanent scar tissue is formed and is called a foreign body capsule (FBC). During this second stage, the local, pH returns to more neutral physiologic levels. While a variety of controlled drug delivery methods exist, they do not account for biological variations or mechanically traumatic events to implanted devices beyond the initial formation of the FBC. They control the release of biologically active agents versus time but are not necessarily responsive to the environment in which they are placed.

### SUMMARY

The present technology relates to new drug delivery systems and methods, and implantable devices which improve vascularization of the device and/or substantially reduce or suppress adverse biological responses by selectively modulating, activating, or deactivating delivery of a bioactive molecule, under physiological conditions. Thus, the systems and devices may be designed to respond to the local biological environments in which they are placed. Accordingly, in one aspect, the present technology provides a drug delivery system including an implantable medical device configured to include a biointerface comprising a polymer and a bioactive molecule attached to the polymer via a silyl ether linker.

In one embodiment, the silyl, ether linker has the formula

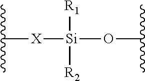

wherein
X links the silyl ether to the polymer and is selected from a covalent bond, oxygen, or an alkylene, alkylene ether, alkylene polyether, alkenylene, or siloxane group;

R₁ and R₂ are independently selected from —H, or a substituted or unsubstituted alkyl, cycloalkyl, alkoxy, alkenyl, quaternary aminoalkyl, aryl, aralkyl, or heterocyclylalkyl group; and the silyl ether oxygen is attached to the bioactive molecule. In some embodiments, R₁ and R₂ are independently selected from substituted or unsubstituted methyl, ethyl, isopropyl, tert-butyl, methoxy, ethoxy, aminomethyl, aminopropyl, trimethylamino propyl, 4,5-dihydroimidazolyl propyl, carboxymethyl, carboxypropyl, phenyl, or benzyl groups. In an illustrative embodiment, the silyl ether linker is 3-aminopropyl methoxy silyl ether.

In some embodiments, the bioactive molecule is selected from a group consisting of anti-inflammatory agents, angiogenic molecules, anti-infective agents, anesthetics, growth factors, adjuvants, wound factors, resorbable device components, immunosuppressive agents, antiplatelet agents, anticoagulants, ACE inhibitors, cytotoxic agents, anti-barrier cell compounds, vascularization compounds, and anti-sense molecules. In some embodiments, the bioactive molecule is selected from the group consisting of monobutyrin, S1P (sphingosine-1-phosphate), cyclosporin A, anti-thrombospondin-2, rapamycin (and its derivatives), and dexamethasone. In some embodiments, the bioactive molecule is a small bioactive molecule. In an illustrative embodiment, the bioactive molecule is an anti-inflammatory agent or an angiogenic molecule.

In some embodiments, the polymer at least partially or completely coats the implantable medical device to form the biointerface membrane. In some embodiments, the polymer coating forms a porous biointerface membrane.

In some embodiments the polymer is a block copolynmer, a random copolymer, a graft copolymer and a biostable polymer. In. one embodiment, the coating comprises a polymer selected from the group consisting of silicone, polyurethane, polytetrafluoroethylene, polyethylene-co-tetrafluoroethylene, polyolefin, polyester, and polycarbonate, polylactone, polyamide, and polyacrylate. In an illustrative embodiment, the polymer is silicone.

In one embodiment, the silyl ether linker is hydrolyzable at a pH of less than 7. In an illustrative embodiment, the silyl ether linker is hydrolyzable at acidic pH. In an illustrative embodiment, the silyl, ether linker is hydrolyzable at the physiological pH of a wound healing environment.

In one embodiment of the present drug delivery system, the implantable medical device is at least partially coated with silicone, and the silicone is linked to the bioactive molecule via a silyl ether linker. In an illustrative embodiment, the bioactive molecule is monobutyrin.

In one embodiment, the silyl ether linker and the bioactive molecule have the structure:

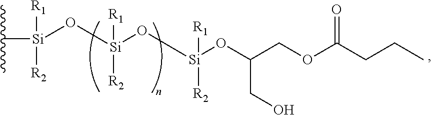

wherein
R₁ and R₂ are independently selected from —H or a substituted or unsubstituted alkyl, cycloalkyl, alkoxy, alkenyl, quaternary aminoalkyl, aryl, aralkyl, or heterocyclylalkyl group; and
n is an integer from 0 to 20.

In one embodiment, the implantable medical device is selected from a stent, glucose sensor, ocular implant, breast implant, penile implant, cosmetic implant, orthopedic implant, and cardioverter-defibrilator.

In another aspect, the present technology provides a method including releasing a bioactive molecule from any of the drug delivery systems described herein at a pH of less than 7. In one embodiment of the method, the bioactive molecule is an anti-inflammatory agent or an angiogenic molecule. In some embodiments, the medical device is implanted in a host.

The foregoing summary is illustrative only and is not intended to be in any way limiting. In addition to the illustrative aspects, embodiments and features described above, further aspects, embodiments and features will become apparent by reference to the following drawings and the detailed description.

### BRIEF DESCRIPTION OF THE DRAWINGS

FIGS. 1A-1C illustrate various embodiments of the design for a drug delivery system according to the present technology.

FIG. 2 depicts a porous silicone biointerface loaded with a silyl ether linked drug.

### DETAILED DESCRIPTION

In the following detailed description, reference is made to the accompanying figures which form a part hereof. In the figures, similar symbols typically identify similar components, unless context dictates otherwise. The illustrative embodiments described in the detailed description, figures and claims are not meant to be limiting. Other embodiments may be utilized, and other changes may be made, without departing from the spirit or scope of the subject matter presented here.

The present technology is described herein using several definitions, as set forth throughout the specification. As used herein, unless otherwise stated, the singular forms “a,” “an,” and “the” include plural reference. Thus, for example, a reference to “a cell” includes a plurality of cells, and a reference to “a molecule” is a reference to one or more molecules.

As used herein, “about” will be understood by persons of ordinary skill in the art and will vary to some extent depending upon the context in which it is used. If there are uses of the term which. are not clear to persons of ordinary skill, in. the art, given the context in which it is used, “about” will mean up to plus or minus 10% of the particular term.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted. group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones azides; amides; ureas; amidines; guanidines; enamines; imides; isocyanates; isothiocyanates; cyanates; thiocyanates; imines; nitro groups nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having the number of carbons indicated herein. In some embodiments, an alkyl group has from 1 to 12 carbon atoms, 1 to 1.0 carbon atoms, from 1 to 8 carbons or, in some embodiments, from 1 to 6, or 1, 2, 3, 4 or 5 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl isopentyl, and 2,2-dimethylpropyl groups. In some embodiments, representative substituted alkyl groups may be substituted one or more times with substituents such as those listed above and include, without limitation, haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Quaternary aminoalkyl groups include alkyl groups substituted with a quaternary amino group. As used herein, a quaternary amino group has a total of four substituents including the alkyl to which it is bound. Thus quaternary amino groups bear a positive charge and exist as a salt with a negatively charged counterion. In addition to the alkyl group, the substituents on the quaternary amino may be the same or different and may include any of alkyl, alkenyl, aryl, arylalkyl, heterocyclyl and heterocyclylalkyl groups.

Cycloalkyl groups include mono-, bi- or tricyclic alkyl groups having from 3 to 12 carbon, atoms in the ring(s), or, in some embodiments, 3 to 10, 3 to 8, or 3 to 4, 5, or 6 carbon atoms. Exemplary monocyclic cycloalkyl groups include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments, the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Alkenyl groups include straight and branched chain alkyl groups as defined above, except that at least one double bond exists between two carbon atoms. Thus, alkenyl groups have from 2 to 12 carbon atoms, and typically from 2 to 10 carbons or, in some embodiments, from 2 to 8, 2 to 6, or 2 to 4 carbon atoms. Examples include, but are not limited to, vinyl, allyl, CH═CH(CH₃), CH═C(CH₃)₂, C(CH₃)═CH₂, C(CH₃)═CH(CH₃), C(CH₂CH₃)═CH₂, among others. Representative substituted alkenyl groups may be mono-substituted or substituted more than once, such as, but not limited to, mono-, di- or tri-substituted with substituents such as those listed above.

The term “alkylene” as used herein, refers to a divalent saturated branched or unbranched hydrocarbon chain containing from 1 to 12 carbon atoms, and includes, for example, methylene, ethylene, propylene, 2-methylpropylene, hexylene and the like. In some embodiments, the term includes lower alkylene, i.e., an alkylene group of 1 to 6, or even 1 to 4, carbon atoms. In other embodiments, the term includes cycloalkylene groups which refers to a divalent cyclic alkyl group. In some embodiments, the cycloalkylene group is a 5- or 6-member ring.

Alkylene ethers and alkylene polyethers include alkylene groups that include respectively, one or more ether oxygen atoms. Thus, alkylene ethers and polyethers include, e.g., —CH₂CH₂O—, —CH(OCH₃)CH₂—, —CH₂CH₂OCH₂—, and —[CH₂CH₂O]₂—.

As used herein, the term “alkenylene” refers to a straight or branched chain divalent hydrocarbon radical having, from 2 to 1.2 carbon atoms and one or more carbon-carbon, double bonds, including but not limited to vinylene, allylene, 2-butenylene, and the like.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl, or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include, but are not limited to, benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups.” Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothriazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, piperazin-1-yl-methyl, tetrahydrofuran-2-yl-ethyl, and piperidinyl-propyl. Representative substituted heterocyclylalkyl groups may be substituted one or more times with substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include, but are not limited to, methoxy, ethoxy, propoxy, butoxy, pentoxy, hexoxy, and the like. Examples of branched alkoxy groups include, but are not limited to, isopropoxy, sec-butoxy, tert-butoxy, isopentoxy, isohexoxy, and the like. Examples of cycloalkoxy groups include, but are not limited to, cyclopropyloxy, cyclobutyloxy, cyclopentyloxy, cyclohexyloxy, and the like. Representative substituted alkoxy groups may be substituted one or more times with substituents such as those listed above.

The term “amine” (or “amino”) as used herein refers to —NR³⁵R³⁶ groups, wherein R³⁵ and R³⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino. The term “alkylamino” is defined as —NR³⁷R³⁸, wherein at least one of R³⁷, and R³⁸ is alkyl and the other is alkyl or hydrogen. The term “arylamino” is defined as —NR³⁹R⁴⁰, wherein at least one of R³⁹ and R⁴⁰ is aryl and the other is aryl or hydrogen.

The term “carboxylate” as used herein refers to a —COOH group.

The term “ester” as used herein refers to —COOR³⁰ groups, R³⁰ is a substituted or unsubstituted alkyl, cycloalkyl, alkenyl, alkynyl, aryl, aralkyl, heterocyclylalkyl or heterocyclyl group as defined herein.

The term “amide” (or “amido”) includes C- and N-amide groups, i.e., —C(O)NR³¹R³², and —NR³¹C(O)R³² groups, respectively. R³¹ and R³² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR³¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR³³C(O)OR³⁴ and —OC(O)NR³³R³⁴ groups, respectively. R³³ and R³⁴ are independently a substituted or unsubstituted alkyl, alkenyl, alkynyl, cycloalkyl, aryl, aralkyl, heterocyclylalkyl, or heterocyclyl group as defined herein. R³³ may also be H.

The term “sulfonamido” includes S- and N-sulfonamide groups, i.e., —SO₂NR³⁸R³⁹ and —NR³⁸SO₂R³⁹ groups, respectively. R³⁸ and R³⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while sulfides include —SR⁴⁰ groups, sulfoxides include —S(O)R⁴¹ groups, sulfones include —SO₂R⁴² groups, and sulfonyls include —SO₂OR⁴³, R⁴⁰, R⁴¹, R⁴², and R⁴³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁴⁴—C(O)—NR⁴⁵R⁴⁶ groups. R⁴⁴, R⁴⁵, and R⁴⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁴⁷)NR⁴⁸R⁴⁹ and —NR⁴⁷C(NR⁴⁸)R⁴⁹, wherein R⁴⁷, R⁴⁸, and R⁴⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁵⁰C(NR⁵¹)NR⁵²R⁵³, wherein R⁵⁰, R⁵¹, R⁵² and R⁵³ are each independently hydrogen, or a substituted or unsubstituted alkyl, cycloalkyl, alkenyl, alkynyl, aryl aralkyl, heterocyclyl or heterocyclylalkyl, group as defined herein.

The term “enamine” refers to —C(R⁵⁴)═C(R⁵⁵)NR⁵⁶R⁵⁷ and —NR⁵⁴C(R⁵⁵)═C(R⁵⁶)R⁵⁷, wherein R⁵⁴, R⁵⁵, R⁵⁶ and R⁵⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen.” or “halo” as used herein refers to bromine, chlorine, fluorine, or iodine. In some embodiments, the halogen is fluorine. In other embodiments, the halogen is chlorine or bromine.

The term “hydroxy’ as used herein can refer to —OH or its ionized form, —O⁻.

The term “imide” refers to —C(O)NR⁵⁸C(O)R⁵⁹, wherein R⁵⁸ and R⁵⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR⁶⁰(NR⁶¹) and —N(CR⁶⁰R⁶¹) groups, wherein R⁶⁰ and R⁶¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R⁶⁰ and R⁶¹ are not both simultaneously hydrogen.

The term “nitro” as used herein refers to an —NO₂ group.

The term “siloxane group” as used herein refers to organic groups containing one or more siloxy subunits of formula —Si(R₁)(R₂)—O—. R₁ and R₂ are independently selected from —H, or a substituted or unsubstituted alkyl, cycloalkyl, alkoxy, alkenyl, quaternary aminoalkyl, aryl aralkyl, or heterocyclylalkyl group. In some embodiments, the siloxane include from 1, 2, 3, 4, 5, 6, 7, 8, 9, or 10 siloxy subunits.

The term “trifluoromethyl” as used herein refers to —CF₃.

The term “trifluoromethoxy” as used herein refers to —OCF₃.

As used herein, the “administration” of an agent, drug, or peptide to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The term “biostable” as used herein, describes, without limitation, the property of being resistant to degradation by processes that are encountered in vivo. Thus, a biostable material may be a polymer that is resistant to degradation in vivo, such as a polymer resistant to homolytic cleavage of the polymer backbone. Biostable materials are typically stable over for the life of the device. Non-limiting examples include 1 year for a glucose sensor and 20 years for pacemaker leads. Illustrative examples of such biostable polymers include medical grade silicone rubber and polyurethane.

As used herein, an “implantable medical device” refers to any type of appliance that is totally or partly introduced, surgically or medically, into a subject's body or by medical intervention into a natural orifice, and which is intended to remain there after the procedure. The duration of implantation may be essentially permanent, i.e., intended to remain in place for the remaining lifespan of the subject, or temporary, until the device biodegrades or until it is physically removed. Examples of implantable medical devices include, without limitation: implantable cardiac pacemakers and defibrillators; leads and electrodes for the preceding; implantable organ stimulators such as but not limited to nerve, bladder, sphincter and diaphragm stimulators, cochlear implants, prostheses, vascular grafts, self-expandable stents, balloon-expandable stents, stent-grafts, grafts, artificial heart valves and cerebrospinal fluid shunts. In some embodiments, implantable medical devices may include breast and penile implants, cosmetic or reconstructive implants, devices for cell transplantation, drug delivery devices, and electrical signalling or delivery devices. An implantable medical device specifically designed and intended solely for the localized delivery of a therapeutic agent is within the scope of the present technology.

As used herein, the term “therapeutically effective amount” refers to a quantity sufficient to achieve a desired therapeutic and/or prophylactic effect, e.g., an amount which results in the prevention of, or a decrease in, the symptoms associated with inflammation due to wound healing. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination, with one or more additional therapeutic compound. In the methods described herein, the vitreous substitute may be administered to a subject having one or more signs or symptoms of an ophthalmic condition. For example, a “therapeutically effective amount” of an anti-inflammatory drug is an amount at which the response to an inflammatory event or source of inflammation (e.g., an implanted medical device), is at a minimum, ameliorated.

By “subject,” is meant any animal, that can benefit from the administration of the disclosed devices. Thus, subjects includes mammals, e.g., a human, a primate, a dog, a cat, a horse, a cow, a pig, or a rodent, e.g., a rat or mouse. In some embodiments, the subject is a human. The subjects may be normal, healthy subjects or subjects having, or at risk for developing, a particular biological disease or condition. By way of example only, the subject may be a subject having, or at risk for developing, foreign body reaction upon implantation of a medical device.

As used herein, the term “bioactive molecule” refers to a molecule that is capable of forming a covalent bond with the silyl ether linker, and exhibits biological activity in an animal. In some embodiments, the bioactive molecule is a small bioactive molecule and has a molecular weight of less than about 1500 g/mole. Bioactive molecules include, without limitation, drugs, prodrugs, vitamins, and cofactors.

Disclosed herein are methods and apparatuses for delivery of bioactive molecules. This disclosure is drawn, inter alia, to drug delivery systems which include an implantable medical device. Further disclosed herein are drug delivery systems which reduce or suppress adverse biological responses associated with implantable devices. In one aspect, the drug delivery systems promote vascularization in tissues surrounding the implanted device. In another aspect, these systems can be designed to vary the rate of delivery of bioactive molecules with a change in the physiological environment surrounding the device. The systems disclosed herein can be used to deliver a wide variety of bioactive molecules. The systems and devices of present technology provide a cost-effective, efficient way for the sustained delivery of a variety of bioactive molecules without having to tailor the materials or the design of the device to complement the particular physical and chemical properties of each drug or bioactive molecule, as well as the surrounding physiological environment.

Thus, in one aspect, the present disclosure provides a drug delivery system comprising an implantable medical device configured to include a biointerface comprising a polymer and a bioactive molecule attached to the polymer via a silyl ether linker.

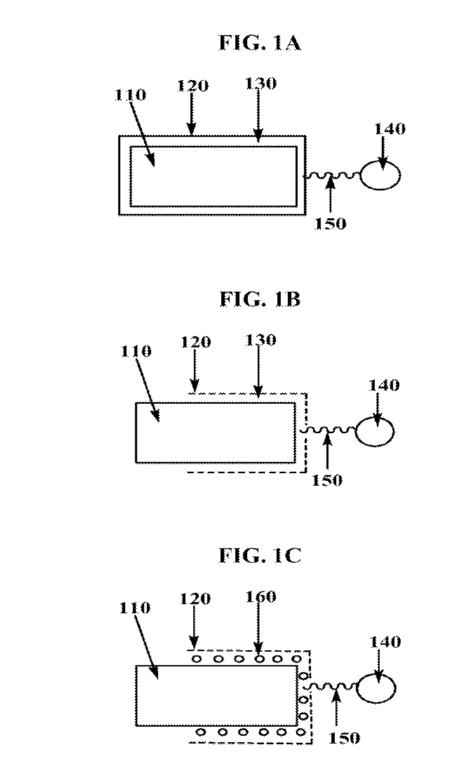

FIG. 1A illustrates one embodiment of a drug delivery system of the present technology. The drug delivery system has an implantable medical device 110 which has a biointerface 120 which contacts the tissue into which the medical device is implanted. The biointerface includes one or more polymers 130 and may be a biointerface membrane. The polymer is attached to one or more bioactive molecules 140 via a silyl ether link 150. In vive the silyl ether linkage is cleaved to release the bioactive molecule. In some embodiments (as shown in FIG. 1B), the polymer 130 at least partially coats the implantable medical device 110. In some embodiments (as shown in FIG. 1C), the polymer coating forms a porous biointerface membrane 160.

Examples of the implantable medical device include the entire spectrum of articles adapted for medical use, including analyte measuring devices, cell transplantation devices, drug delivery devices, electrical signal delivery and measurement devices, stents, diagnostic devices such as glucose monitors, artificial organs such as artificial hearts and artificial kidneys, orthopedic implants, pins, and plates, catheters and other tubes including urological and biliary tubes, endotracheal tubes, central venous catheters, dialysis catheters, pulmonary catheters, and urinary catheters, urinary devices, shunts, prostheses including breast implants, penile implants, cosmetic implants, vascular grafting prostheses, heart valves, artificial joints, artificial larynxes, otological implants, pacemakers and implantable cardioverter-defibrillators, and the like. Other examples of implantable devices will be readily apparent to practitioners in these arts. In an illustrative embodiment, the implantable medical device is selected from a stent, glucose sensor, breast implant, penile implant, cosmetic implant, orthopedic implant, and cardioverter-defibrilator.

The biointerface of the present technology may take several forms. In some embodiments, the implantable medical device may be constructed in whole or in part from the polymer including a bioactive molecule attached to the polymer via a silyl ether linker. For example, finger joint implants may be constructed entirely of silicone rubber, and a bioactive molecule may be attached to the surface via a silyl ether bond as described herein. In some embodiments, the polymer at least partially or completely coats the implantable medical device to form a biointerface membrane that may be, e.g., from about 0.1 mm to about 3 mm thick. Examples of suitable thicknesses for such membranes include about 0.1 mm, about 0.2 mm, about 0.3 mm, about 0.4 mm, about 0.5 mm, about 0.6 mm, about 0.8 mm, about 1 mm, about 1.2 mm, about 1.5 mm, about 1.8 mm, about 2 mm, about 2.5 mm, about 3 mm, and values between any two of these. The term “biointerface membrane,” as used herein, refers to a permeable or semi-permeable membrane that functions as a device-tissue interface from which the bioactive molecule is released. In an illustrative embodiment, the device is a glucose sensor fashioned with an epoxy polymer body. The device body is wrapped in a medical grade silicone rubber about 1 mm thick and is permeable to glucose. About 5-10% by volume is loaded with a bioactive molecule using the silyl ether linkage of the present technology.

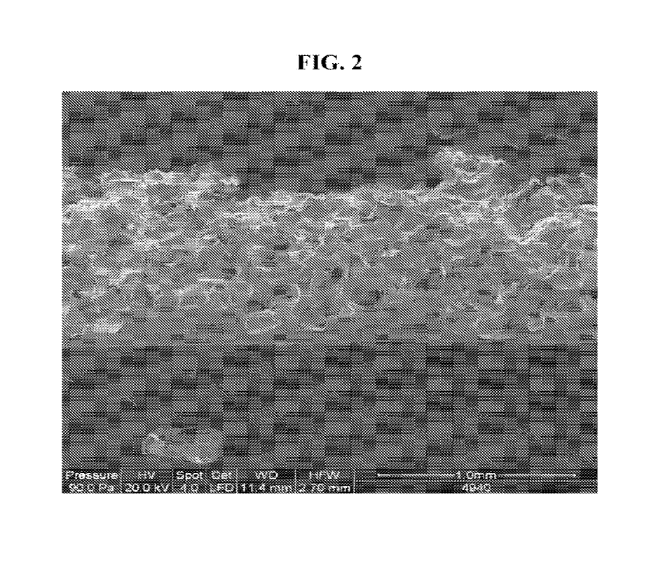

In some embodiments, the polymer coating forms a porous biointerface membrane. (See, e.g., FIG. 2.) Such porous membranes have cavities that the surrounding tissue may grow into and anchor the device in place. In some embodiments, the biointerface membrane is composed of two domains. The first domain supports tissue in growth, interferes with barrier cell layer formation, and includes an open cell configuration having cavities and a solid portion. The second domain is resistant to cellular attachment and impermeable to cells (e.g., macrophages). Examples of biointerface membranes include, but are not limited to those disclosed in U.S. Pat. Nos. 7,364,592; 7,192,450; 7,134,999; and 6,702,857, each incorporated herein by reference in its entirety. In any such embodiments, the bioactive molecule may be a small bioactive molecule.

Polymers useful in the present technology include those known to be suitable for use in vivo, e.g., with medical implants, and will be readily apparent to one skilled in the art. In an illustrative embodiment, the polymers may include silicone, polytetrafluoroethylene, polyethylene-co-tetrafluoroethylene, polyolefin, polyester, polycarbonate, biostable polytetrafluoroethylene, homopolymers, copolymers, polyurethanes, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), cellulosic polymers, polysulfones and block copolymers thereof including, for example, di-block, tri-block, alternating, random and graft copolymers or any biostable polymer known in the art such as polyurethane and a hydrophilic polymer or polyurethane and polyvinylpyrrolidone. In some embodiments, the polymer is selected from the group consisting of silicone, polyurethane, polytetrafluoroethylene, polyethylene-co-tetrafluoroethylene, polyolefin, polyester, and polycarbonate, polylactone, polyamide, and polyacrylate. In an illustrative embodiment, the polymer is silicone.

Various methods known in the art can be used to link the polymer to the silyl ether linker. For example, after the bioactive molecule is covalently bonded to the silyl ether linker to form a prodrug-like conjugate, the conjugate may be cross-linked, and cured into the bulk of a medical-grade silicone rubber and applied to the surface of an implantable medical device as a coating or a porous biointerface. In some embodiments, a silicone rubber is used which includes functionalized polydimethylsiloxane (PDMS). The terminal ends of such polymers may contain vinyl, silanol or other reactive functional groups suitable for cross-link formation. A crosslinking molecule that possesses functional groups matched for reaction with the PDMS is then added to the mixture of conjugate and silicone rubber to form the cross-linked network. In an illustrative embodiment, the cross-linker for a vinyl terminated PDMS would contain three or more silicon-hydrogen groups that may be chemically added to the vinyl group using a platinum catalyst. Alternatively, a silanol terminated PDMS can be cross-linked using triacetoxymethylsilane or triacetoxyethylsilane using a dibutyltin dilaurate catalyst. In this system the cross-linking occurs through condensation of the silanol end groups with the acetoxysilane to afford a siloxane bond and one molecule of acetic acid. For example, a silane comprising a bioactive molecule (including without limitation, a small bioactive molecule) linked through a silyl ether bond and having at least one acetoxy group may be blended into a tin-alkoxy cure silicone such as NuSil DDU-4340 or DDU-4351 and coated and cured on any suitable implantable device. In each of these cross-linking systems, a bioactive molecule covalently bonded through a silyl ether linker can be incorporated into the overall silicone rubber by way of the appropriate functional groups on the linker. Thus, any polymer material can be linked to the silyl ether linker of the bioactive molecule and this preloaded polymer can then be coated, sprayed or attached to the surface of the medical device to be implanted using suitable methods known in the art.

In some embodiments, the polymer is linked to silyl ether which, in turn, is linked to the bioactive molecule (e.g. a small bioactive molecule). Thus, in some embodiments, the link between the polymer and the silyl ether is selected from a covalent bond, oxygen, an alkylene, alkylene ether, alkylene polyether, alkenylene, or siloxane group.

The bioactive molecule may be covalently attached to the polymer of the implantable medical device via a silyl ether linkage. Methods of synthesis of silyl ethers of bioactive molecules are known in the art. For example, bioactive molecules which contain an alcoholic group may easily be reacted with silane compounds (including but not limited to halosilanes) to form a silyl ether linkage. In some embodiments, the bioactive molecule may be modified to include a functional group capable of forming a covalent bond with the silyl linker. Suitable functional groups as well as suitable conditions for forming such a bond are well known in the art. For example, numerous functional groups which form a covalent bond with silyl groups as protecting groups are described in T. W. Greene and G. M. Wuts, Protecting Groups in Organic Synthesis, 3rd Edition, Wiley, New York (1999), and references cited therein.

In an illustrative embodiment, attachment of a small bioactive molecule, such as monobutyrin, with a silyl ether linker and an implantable drug device can be represented by the following scheme.

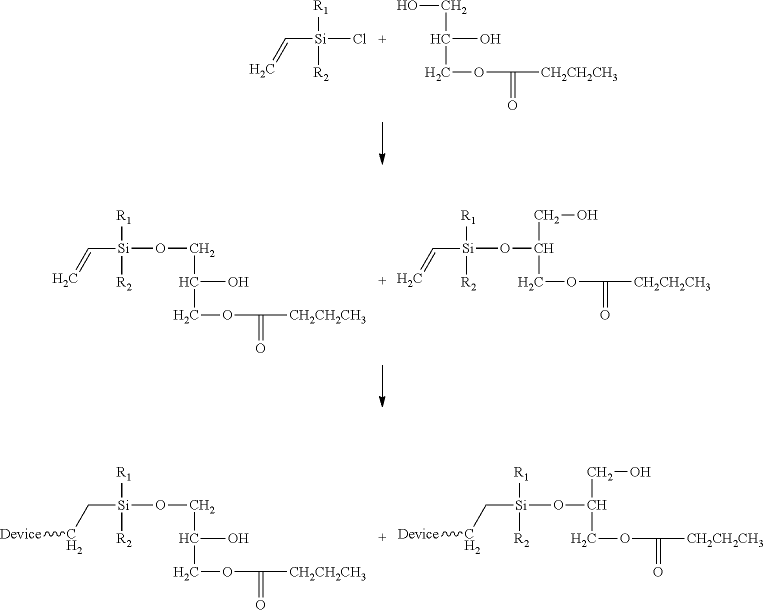

In the scheme, a vinyl silyl halide such as, e.g., a vinyl silyl chloride is reacted with monobutyrin, under, e.g., basic conditions. Since monobutyrin has two hydroxyl groups, two isomers of the silylated drug result. The silylated monobutyrin may then be attached to the polymer through reaction at the vinyl group.

A wide variety of bioactive molecules can be delivered using the present technology. In some embodiments, the bioactive molecule is selected from a group consisting of anti-inflammatory agents, angiogenic molecules, anti-infective agents, anesthetics, growth factors, adjuvants, wound factors, resorbable device components, immunosuppressive agents, antiplatelet agents, anticoagulants, ACE inhibitors, cytotoxic agents, anti-barrier cell compounds, vascularization compounds, and anti-sense molecules. Anti-inflammatory agents that may be used in the present technology include but are not limited to steroids and non-steroidal agents (e.g., dexamethasone, prednisolone, aspirin, acetaminophen, ibuprofen, naproxen, piroxicam). Angiogenic molecules include but are not limited to sphingosine-1-phosphate and monobutyrin. Immunosuppressive agents include but are not limited to cyclosporin. A, rapamycin and its derivatives such as CCI-779, RAD001 and AP23576. In some embodiments, the bioactive molecule is selected from the group consisting of monobutyrin, S1P (sphingosine-1-phosphate), cyclosporin A, anti-thrombospondin-2, rapamycin (and its derivatives), and dexamethasone. In an illustrative embodiment, the bioactive molecule is an anti-inflammatory agent or an angiogenic molecule. In some embodiments, the bioactive molecule is a small bioactive molecule such as, but not limited to, monobutyrin.

In one embodiment, the silyl ether linker has the formula

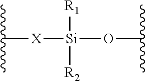

wherein
X links the silyl ether to the polymer and is selected from a covalent bond, oxygen, or an alkylene, alkylene ether, alkylene polyether, alkenylene, or siloxane group;
R₁ and R₂ are independently selected from —H, or a substituted or unsubstituted alkyl, cycloalkyl, alkoxy, alkenyl, quaternary aminoalkyl, aryl, aralkyl, or heterocyclylalkyl group; and the silyl ether oxygen is attached to the bioactive molecule. In some embodiments, R₁ and R₂ are independently selected from substituted or unsubstituted methyl, ethyl, isopropyl, tert-butyl, methoxy, ethoxy, aminomethyl, aminopropyl, trimethylamino propyl, 4,5-dihydroimidazolyl propyl, carboxymethyl, carboxypropyl, phenyl, or benzyl groups. In an illustrative embodiment, the silyl ether linker is 3-aminopropyl methoxy silyl ether.

In one embodiment, the implantable medical device is at least partially coated with silicone, and the silicone is linked to a bioactive molecule via a silyl ether linker. In an illustrative embodiment, the bioactive molecule is a small bioactive molecule such as, without limitation, monobutyrin.

In one embodiment, the silyl ether linker and bioactive molecule have the structure:

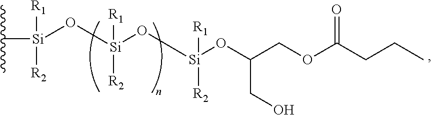

wherein
R₁ and R₂ are independently selected from —H, or a substituted or unsubstituted alkyl, cycloalkyl, alkoxy, alkenyl, quaternary aminoalkyl, aryl, aralkyl, or heterocyclylalkyl group; and
n is an integer from 0 to 20.

A feature of the present technology is the ability to tune the silyl ether linker of the drug delivery system to be responsive to the changing physiological conditions to which the system is exposed. Specifically, by changing the substituents on the silicon atom of the silyl ether linkage, the sensitivity of the silyl ether linkage to hydrolysis may be adjusted. Thus, the rate of cleavage of the silyl ether linkage and release of the bioactive molecule will vary with the pH of the physiological environment surrounding the implant. In some embodiments, the silyl ether linker is readily hydrolyzed under physiological pH conditions, e.g., 7.3 to 7.5. In some embodiments, the silyl ether linker is hydrolyzable under acidic conditions, e.g., a pH ranging from about 4.5 to less than 7. In some embodiments, the silyl ether linker is hydrolyzable under neutral conditions, e.g., at about pH 7. In an illustrative embodiment, the silyl ether linker is hydrolyzable at a pH of less than 7. The substitution on both the silicone atom and the alcohol carbon of the bioactive molecule can affect the rate of hydrolysis due to steric and electronic effects. This allows for the possibility of tuning the rate of hydrolysis of the silicone-oxygen-carbon linkage by changing the substitution on either the organosilane, the alcohol, or both the organosilane and alcohol to facilitate the desired affect. In addition, charged or reactive groups, such as amines or carboxylate, may be linked to the silicone atom, which confers the labile compound with charge and/or reactivity.

As an illustration of this feature, in some embodiments of the present technology, the silyl ether linkage between the implantable medical device and the pro-drug of the bioactive molecule can be represented as

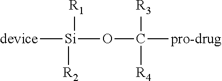

The covalent silyl ether connection between the device and drug can be hydrolyzed at depressed or lower pH levels that are present during active wound healing. A aspect of the present technology is the ability to tune the pH for which the connection is cleavable by selection of the substituent groups on the silicone atom. In the above bioactive molecule, substituent groups R₁ and R₂ may be chosen to influence the electronic nature of the silyl ether bond. For example, the silyl ether bond will be more sensitive to acid catalyzed cleavage if R₁ and R₂ are methyl than if they are ethyl. In addition, the substituent groups R₃ and R₄ on the bioactive molecule may also be changed in order to tune the cleavage of the linker, provided that this change does not dramatically alter the therapeutic effect of the bioactive molecule. However, generally the structure of the bioactive molecule will be left undisturbed beyond conjugating to the silyl group. In some embodiments, the bioactive molecule is a small bioactive molecule.

Thus, in another aspect, the present technology provides a method comprising releasing a bioactive molecule from a drug delivery system, described herein, at a pH of less than 7. In some embodiments of the method, the bioactive molecule is released at a pH of about 3 to about 7. In some embodiments of the method, the bioactive molecule is released at a pH of about 4 to about 6. In some embodiments of the method, the bioactive molecule is released at a physiological pH of a wound healing environment. In one embodiment of the method, the bioactive molecule is an anti-inflammatory agent or an angiogenic molecule. In an illustrative embodiment of the method, the bioactive molecule is a small bioactive molecule, such as, without limitation, monobutyrin.

In some embodiments, the medical device of the present technology is implanted in a host. The host can be any suitable subject in need of an implant such as humans and other mammals. In an illustrative embodiment, the host is a human. To deliver the bioactive molecule to a specific body region, the drug delivery device including the implantable medical device of the present technology can be guided into a position in the desired region to be treated, using conventional techniques. After positioning the device, the device comes into contact with the surrounding tissue. The physiological conditions around the device cause the silyl ether linker to be hydrolyzed resulting in a sustained release of the bioactive molecule into the surrounding tissue. In some embodiments a therapeutically effective amount of the bioactive molecule is released, e.g., an amount effective to reduce the host inflammatory response to the implanted medical device.

The present invention, thus generally described, will be understood more readily by reference to the following examples which are provided by way of illustration and are not intended to be limiting of the present invention.

### EXAMPLES

The present disclosure is further illustrated by the following examples which should not be construed as limiting in any way.

### Example 1

### Synthesis of Silyl Ether of a Bioactive Molecule

Monobutyrin (40 mmol), imidazole (48 mmol) and dichloromethane (90 mL) are added to a 250 mL three-necked round-bottomed flask. The flask is fitted with a Teflon-coated magnetic stir bar, a gas inlet, a thermometer and a 150 mL pressure-equalizing dropper funnel. The apparatus is flushed with nitrogen, and maintained under a slight positive pressure throughout the reaction. The dropper funnel is then charged with the chloromethoxydimethylsilane (44 mmol) and dichloromethane (10 mL). After this, the silane is added to the reaction flask drop wise while maintaining the reaction mixture's temperature no greater than 30° C. After complete addition, the reaction is allowed to stir for an additional hour. After this time, the reaction mixture is transferred to a 250 mL separator funnel and washed three times with 60 mL of distilled water. Dichloromethane is removed from the crude product under reduced pressure. The compound may be purified via distillation under reduced pressure and characterized, e.g., by ¹H NMR and mass spectroscopy.

### Example 2

### Synthesis of Polymer Linked to the Silyl Ether Linker/Bioactive Molecule Conjugate

The silyl ether linker/bioactive molecule conjugate can be covalently incorporated into the bulk of a silicone material. The compound from Example 1 is blended into a tin-alkoxy cure silicone such as NuSil DDU-4340 or DDU-4351. The compound will create a siloxane bond to the silicone through the methoxy group on the conjugate. For example, 1 part of the conjugate compound is thoroughly mixed into 100 parts of NuSil DDU-4340 and cast into the desired shape. This mixture is allowed to cure for 30 minutes at room temperature, then removed from its mold. After this time, the article may be further subjected to a post cure treatment for an additional 24 hours at ambient temperature and humidity. In addition to the above example, Table 1 describes how the bioactive molecule conjugate may be incorporated into a silicone article.

| | Formulation | | | |
|---|---|---|---|---|
| Component | 1 | 2 | 3 | 4 |
| Bioactive conjugate from example 1 | 1% | 10% | 10% | 20% |
| NuSil DDU-4340 | 99% | 87.9% | 0 | 0 |
| Polydimethylsiloxane, OH terminated | 0 | 0 | 74.9% | 68.9% |
| Methyltrioximinosilane | 0 | 0 | 5% | 1% |
| Fumed silica, 150 sq.m/g surface area | 0 | 2% | 10% | 10% |
| Dibutyltin dilaurate | 0 | 0.1% | 0.1% | 0.1% |
| Total | 100% | 100% | 100% | 100% |

### Example 3

### Release of Monobutyrin from a Drug Delivery System of the Present Technology

The release of monobutyrin from a drug delivery system of Example 2 may be evaluated using an in vitro assay as follows. After the bioactive conjugate has been formed as described above, the loaded silicone rubber may be placed into glass vials also containing phosphate buffered saline. The pH of the buffer system in duplicate vials is adjusted to 4, 5, 6, 7 and 7.4. The glass vials are stored at normal body temperature (37° C.), and small samples are removed at various time points (e.g., 0.5 h, 1 h, 2 h, 4 h, 8 h, 12 h, 24 h) and subjected to analysis by HPLC. In this way, the release of monobutyrin versus time at various pH levels can be investigated.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like include the number recited and refer to ranges which can be subsequently broken down into subranges as discussed above. Finally, as will be understood by one skilled in the art a range includes each individual member. Thus, for example, a group having 1-3 particles refers to groups having 1, 2, or 3 particles. Similarly, a group having 1-5 particles refers to groups having 1, 2, 3, 4, or 5 particles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein are incorporated by reference in their entireties and for all purposes to the same extent as if each individual publication, patent, or patent application was specifically and individually incorporated by reference in its entirety for all purposes.

1. A drug delivery system comprising an implantable medical device configured to include a biointerface comprising a polymer and a bioactive molecule attached to the polymer via a silyl ether linker.

2. The drug delivery system of claim 1, wherein the silyl ether linker has the formula 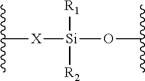
wherein
X links the silyl ether to the polymer and is selected from a covalent bond, oxygen, or an alkylene, alkylene ether, alkylene polyether, alkenylene, or siloxane group;
R₁ and R₂ are independently selected from —H, or a substituted or unsubstituted alkyl, cycloalkyl, alkoxy, alkenyl, quaternary aminoalkyl, aryl, aralkyl, or heterocyclylalkyl group; and
the silyl ether oxygen is attached to the bioactive molecule.

3. The drug delivery system of claim 2, wherein R₁ and R₂ are independently selected from substituted or unsubstituted methyl, ethyl, isopropyl, tert-butyl, methoxy, ethoxy, aminomethyl, aminopropyl, trimethylaminopropyl, 4,5-dihydroimidazolyl propyl, carboxymethyl, carboxypropyl, phenyl, or benzyl groups.

4. The drug delivery system of claim 1, wherein the silyl ether linker is 3-aminopropyl methoxy silyl ether.

5. The drug delivery system of claim 1, wherein the bioactive molecule is selected from a group consisting of anti-inflammatory agents, angiogenic molecules, anti-infective agents, anesthetics, growth factors, adjuvants, wound factors, resorbable device components, immunosuppressive agents, antiplatelet agents, anticoagulants, ACE inhibitors, cytotoxic agents, anti-barrier cell compounds, vascularization compounds, and anti-sense molecules.

6. The drug delivery system of claim 1, wherein the bioactive molecule is selected from the group consisting of monobutyrin, S1P (sphingosine-1-phosphate), cyclosporin A, anti-thrombospondin-2, rapamycin (and its derivatives), and dexamethasone.

7. The drug delivery system of claim 1, wherein the polymer at least partially coats the implantable medical device to form a biointerface membrane.

8. The drug delivery system of claim 7, wherein the polymer coating forms a porous biointerface membrane.

9. The drug delivery system of claim 1, wherein the polymer is selected from the group consisting of silicone, polyurethane, polytetrafluoroethylene, polyethylene-co-tetrafluoroethylene, polyolefin, polyester, polycarbonate, polylactone, polyamide, and polyacrylate.

10. The drug delivery system of claim 1, wherein the polymer is a block copolymer, a random copolymer, a graft copolymer, or a biostable polymer.

11. The drug delivery system of claim 1, wherein the bioactive molecule is an anti-inflammatory agent or an angiogenic molecule.

12. The drug delivery system of claim 1, wherein the bioactive molecule is a small bioactive molecule.

13. The drug delivery system of claim 1, wherein the silyl ether linker is hydrolyzable at a pH of less than 7.

14. The drug delivery system of claim 1, wherein the implantable medical device is at least partially coated with silicone, and wherein the silicone is linked to the bioactive molecule via a silyl ether linker.

15. The drug delivery system of claim 14, wherein the bioactive molecule is monobutyrin.

16. The drug delivery system, of claim 15, wherein the silyl ether linker and bioactive molecule have the structure: 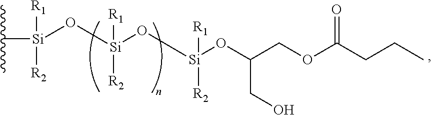
wherein
each R₁ and R₂ is independently selected from —H, or a substituted or unsubstituted alkyl, cycloalkyl, alkoxy, alkenyl, quaternary aminoalkyl, aryl, aralkyl, or heterocyclylalkyl group; and
n is an integer from 0 to 20.

17. The drug delivery system of claim 16, wherein R₁ is an aminopropyl group and R₂ is a methyl group.

18. The drug delivery system of claim 1 wherein the implantable medical device is selected from a stent, glucose sensor, ocular implant, breast implant, penile implant, cosmetic implant, orthopedic implant, and cardioverter-defibrilator.

19. A method comprising releasing a bioactive molecule from a drug delivery system at a pH of less than 7, wherein the drug delivery system comprises an implantable medical device configured to include a biointerface comprising a polymer and a bioactive molecule attached to the polymer via a silyl ether linker.

20. The method of claim 19, wherein the bioactive molecule is an anti-inflammatory agent or an angiogenic molecule.

21. The method of claim 19, wherein the medical device is implanted in a host.
